(19) United States
(12) Patent Application Publication
(10) Pub. No.: US 2019/0272908 A1
(43) Pub. Date: Sep. 5, 2019

(54) SYSTEM AND METHOD FOR DETECTING PHARMACEUTICAL COUNTERFEIT AND FRAUDULENT PRESCRIPTION

(71) Applicant: Tommy Lee Hill, Cedar city, UT (US)

(72) Inventor: Tommy Lee Hill, Cedar city, UT (US)

(21) Appl. No.: 16/289,665

(22) Filed: Mar. 1, 2019

Related U.S. Application Data
(60) Provisional application No. 62/637,729, filed on Mar. 2, 2018.

(51) Int. Cl. G16H 20/10; G06K 9/34; G06T 7/00; H04L 9/06

(52) U.S. Cl. CPC G16H 20/10; G06K 9/344; G06T 7/0012; G06K 2209/05; H04L 9/0643; G06K 2209/01; H04L 2209/38; H04L 9/0637

(57) ABSTRACT

The present invention relates to a system and method for detecting counterfeit, fraudulent and even defectives pharmaceutical and doctor prescription. The system aids the patients to prevent from the potentially deadly Adverse Drug Event (ADE) and Adverse Drug Reaction (ADR) through pill identification, preparation including dose detection and administrative needs by errors due to handwritten prescriptions, instructions, incorrect or fraudulent labeling.

### CROSS-REFERENCE TO RELATED APPLICATION

This application claims the benefit of U.S. Provisional Application No. 62/637,729 filed on Mar. 2, 2018 which is incorporated by reference herein.

### FIELD OF INVENTION

The inventive subject matter relates to the detection of pharmaceutical counterfeit and fraudulent prescription by using vision software, machine learning, and block chain technology.

### BACKGROUND

With the advancement in the technology from day to day life, the data is assessable to masses and anyone can manipulate, use this data for their own benefits. Digitization of the records is good for marketing and product sales but this also leads to a vulnerable situation by providing records to everyone. Some people use this with the consent of the manufacturers while others use the wrongful means to obtain, transform and get benefits from the records, available on the internet while doing forgery with the actual products or records.

Forgery has been a serious issue worldwide. These kinds of malpractices are running in every industry whether it is food, textiles, automobile, or electronics. The most affected among these industries is the pharmaceutical industry. The forged or counterfeit drugs are very dangerous for the health of people and their dosage pose a threat to the life of patients. In the past few years, the number of causalities of patients and reaction to the body is increasing with the increase in the counterfeit drugs in the market.

Various authentication means or procedures including expert verification, labeling, holographic labels, tagging, serial numbers, barcode, RFID tags, and hidden patterns, are adapted to identify the authenticity of various products. All of these methods have an antidote and can be duplicated or tampered with a design around skills resulting in a challenging job for governing bodies to identify any counterfeit or fraudulent item in the supply chain.

The drug manufacturing companies are also facing a huge loss as the brand name of their companies falling down in the market and no one is able to track from where these counterfeit drugs are entering in the market. Unless each unit of custody starting from the manufacturer to the consumer is able to track and identify the manufactured products it seems impossible to track and identify the counterfeit drugs.

In the US Patent Application No. 2007,021,991,6A1 Michael Lucas discloses a method of tracking and authenticating an object through a chain of custody, comprising the steps of; (a) providing a database having client software and residing on at least one storage medium in at least one computer; (b) collecting and storing in said database at least the following data: information relating to said object, information associating said object to an identifier code which is unique to said object, and information relating to user permissions or roles permitting a user to have access to some or all of the information in said database; (c) providing a system for detecting said unique identifier code at multiple locations in said chain of custody; (d) tracking said object in said database by detecting said unique identifier code at each time event in which said object is transferred from one party in the chain of custody to another, or from one location in the chain of custody to another, and collecting and storing the identity of each party who takes custody of said object at each time event; and (e) providing access to information establishing the chain of custody of said object.

In another US Patent Application No. 2018,009,617,5A1 James L. Schmeling et. al discloses a distributed manufacturing platform and related techniques connect designers, manufacturers (e.g., 3D printer owners and other traditional manufacturers), shippers, and other entities and simplifies the process of manufacturing and supplying new and existing products. A distributed ledger or block chain may be used to record transactions, execute smart contracts, and perform other operations to increase transparency and integrity of the supply chain. block chain enabled packaging can be used to track movement and conditions of packages from manufacture, through transit, to delivery.

In yet another US Patent Application No. 2018,013,003,4A1 Benjamin J. Taylor et. al discloses a block chain-based systems and methods incorporate secure wallets; enhanced, randomized, secure identifiers for uniquely identifying discrete items; and cryptographically secure time-stamped block chains. Role-based secure wallets include private cryptographic keys for digitally signing transactions for recording in a block chain. Operations to be performed using role-based wallet can be permitted or restricted based on privileges associated with the respective wallets. Multiple block chains can also be used to track transactions involving different units of account, for example, a measurement of a discrete product being transferred and an associated value of the transaction.

However, there is still a need for a system or a method based on hashing and encryption to detect these counterfeit drugs and alert the authorities including from the manufacturing companies, doctors to patients as well so that the lives of patients can be saved in due time. The inventive subject matter provides a solution to the drawbacks or problems faced related to detection or identification of counterfeit drugs and generate an alarm upon the detection of counterfeit drugs.

### SUMMARY

The inventive subject matter discloses a system for detecting pharmaceutical counterfeit and fraudulent prescription, the system comprising: at least one computing device with a user interface, wherein the at least one computing device is configured to: capture an image of at least one pharmaceutical product; a vision unit communicatively coupled to at least one sensor, wherein the vision unit is configured to capture raw data related to the at least one pharmaceutical product via the at least one sensor; and store the raw data in a storage unit; an object recognition and character recognition (OCR) engine configured to: identify primary data, related to the at least one pharmaceutical product, from the raw data based on at least one predefined parameter; and store the primary data in the storage unit; and an artificial intelligence (AI) module configured to retrieve the raw data and primary data from the storage unit; and generate a first fingerprint data based on each of the primary data and at least one predefined parameter; identify at least one first block that includes the first fingerprint data related to the at least one pharmaceutical product, wherein the at least one pharmaceutical product is available on a networked distributed ledger; and create a second block at every change in custody of the at least one pharmaceutical product on networked distributed ledger, wherein the second block includes the first fingerprint data and a second fingerprint data.

In one objective of the present invention, the AI module is further configured to reject the change in custody of the at least one pharmaceutical product in an event the first fingerprint data is not identified on the networked distributed ledger.

In one another objective of the present invention the AI module is further configured to retrieve the primary data of at least one medical prescription from the storage unit, and compare the primary data of the at least medical prescription with a pre-stored data related to the at least one medical prescription; and authenticate the at least medical prescription based on the comparison of the primary data with the pre-stored data, wherein the pre-stored data comprises at least one anti-counterfeit parameter.

In one another objective of the invention, the AI module is further configured to: identify the at least one medical prescription to be a counterfeit product based on the comparison; and transmit an alert to at least one authority, wherein the alert includes information of last change of custody. The AI module, based on the detection of the counterfeit product, is further configured to generate an alarm on the computing device associated with at least one of a manufacturing unit, a doctor, a pharmacist or a patient.

In one another objective of the invention, the at least one computing device, the vision unit, the OCR engine, and the AI module are communicatively connected to each other.

According to any of the aspect described herein, the AI module is further configured to, create a genesis block and add the first fingerprint data to the genesis block on the networked distributed ledger based on a determination that the first block is unavailable on the networked distributed ledger.

According to any of the aspect described herein, the first fingerprint data is encrypted data.

In one second aspect of the present invention, the change in custody is shifted from at least one of a manufacturing unit to at least one of a pharmacist, a doctor, a nurse, a patient, a technician, or an emergency and law enforcement personnel. The at least one predefined parameter includes a label, a barcode, an image of the at least one product not limited to a pharmaceutical product, a medical prescription, a serial number of a manufacturing unit.

Further, in one objective of the present invention is to provide a system and method for detecting pharmaceutical counterfeit and fraudulent prescription, the method comprising: capturing an image of at least one product; capturing raw data related to the at least one product via at least one sensor; storing the raw data in a storage unit; identifying primary data, related to the at least one product, from the raw data based on at least one predefined parameter; storing the primary data in the storage unit; retrieving the raw data and primary data from the storage unit; generating a first fingerprint data based on each of the primary data and at least one predefined parameter; identifying at least one first block that includes the first fingerprint data related to the at least one product, wherein the at least one product is available on a networked distributed ledger; and creating a second block at every change in custody of the at least one product on networked distributed ledger, wherein the second block includes the first fingerprint data and a second fingerprint data.

### BRIEF DESCRIPTION OF THE DRAWINGS

For a more complete understanding of the inventive subject matter, reference is now made to the following description taken in conjunction with the accompanying drawings, in which:

FIG. 1 is a block diagram representation of the system including a computing device, a scanning module, and Blockchain distributed ledger;

FIG. 2 shows various components of the computing device at a manufacturing unit;

FIG. 3 is a block diagram representation of the interconnection of computing devices of a plurality of user terminals;

FIG. 4 is a flowchart representing the steps of generation of block information on a networked distributed ledger;

FIG. 5 is a flowchart representing the steps of capturing the product related information; and

FIG. 6 is a system representation of one or more interlinked blockchain.

### DETAILED DESCRIPTION OF THE INVENTION

Embodiments of the present disclosure will be described more fully hereinafter with reference to the accompanying drawings and in which example embodiments are shown. Embodiments of the claims may, however, be embodied in many different forms and should not be construed as limited to the embodiments set forth herein. The examples set forth herein are non-limiting examples and are merely examples among other possible examples. The invention can be better understood with the help of a detailed description of the drawings and preferred embodiments of the invention.

As will be appreciated by one skilled in the art, aspects of the present invention may be embodied as a system, method or computer program product. Accordingly, aspects of the present invention may take the form of an entire hardware embodiment, an entire software embodiment (including firmware, resident software, micro-code, etc.), or an embodiment combining software and hardware. Furthermore, aspects of the present invention may take the form of a computer program product embodied in one or more the computer readable medium(s) having computer readable program code embodied thereon.

Any combination of one or more computer readable medium(s) referred to herein as memory or storage unit may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fibre, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer readable signal medium may include a propagated data signal with computer readable program code embodied thereon, for example, in baseband or as part of a carrier wave. Such a propagated signal may take any variety of forms, including, but not limited to, electromagnetic, optical, or any suitable combination thereof. A computer readable signal medium may be any computer readable medium that is not a computer readable storage medium and that can communicate, propagate, or transport a program for use by or in conjunction with an instruction execution system, apparatus, or device.

Program code or software embodied on a computer readable medium may be transmitted using any appropriate medium, including but not limited to wireless, wireline, optical fiber cable, RF and the like, or any suitable combination of the foregoing.

Computer program code for carrying out operations for aspects of the present invention may be written in any combination of one or more programming languages, including an object oriented programming language such as Java, Smalltalk, C++ or the like or conventional procedural programming languages, such as the “C” programming language, AJAX, PHP, HTML, XHTML, Ruby, CSS or similar programming languages. The programming code may be configured in an application, an operating system, as part of system firmware, or any suitable combination thereof. The programming code may execute entirely on the user's computer, partly on the user's computer, as a stand-alone software package, partly on the user's computer and partly on a remote computer or entirely on a remote computer or server as in a client/server relationship sometimes known as cloud computing. In the latter scenario, the remote computer may be connected to the user's computer through any type of network, including a local area network (LAN) or a wide area network (WAN), or the connection may be made to an external computer (for example, through the Internet using an Internet Service Provider).

The term “means” as used herein shall be given its broadest possible interpretation in accordance with 35 U.S.C., Section(s) 112(f) and/or 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, a brief description of the drawings, detailed description, abstract, and claims themselves.

The term “module” such artificial intelligence module as used herein refers to any known or later developed hardware, software, firmware, artificial intelligence, fuzzy logic, machine learning algorithm, deep learning algorithm or combination of hardware and software that is capable of performing the functionality associated with that element. The module can consist of a software agent, a fuzzy logic algorithm, a predictive algorithm, an intelligence rendering algorithm, object recognition module and optical character recognition and a self-learning (including relearning) algorithm. It should be noted that the self-learning (including relearning) algorithm can include a self-learning artificial intelligence algorithm and/or a self-learning neural network algorithm and/or a quantum computer enhanced machine learning algorithm.

The terms “determine”, “calculate” and “compute,” and variations thereof, as used herein, are used interchangeably and include any type of methodology, process, mathematical operation or technique.

The term “computing device” described herein below refers to any processing device, and may include mobile phones, smartphones or PDAs, Tablet, Kiosk computing devices and the like. In one embodiment, a computing device is a touchscreen device for receiving an input from a user via touch, voice, gesture and other computing devices.

The term “item” described herein below refers to any product, goods, article, food, electronic circuit or IC chips and may include any product which uses as a commodity and sell/purchased/manufactured.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks. As used herein, a “terminal” or node or computing device should be understood to be any one of a general purpose computer, as for example a personal computer or a laptop computer, a client computer configured for interaction with a server, a special purpose computer such as a server, or a smartphone, softphone, tablet computer, personal digital assistant or any other machine adapted for executing programmable instructions in accordance with the description thereof set forth above.

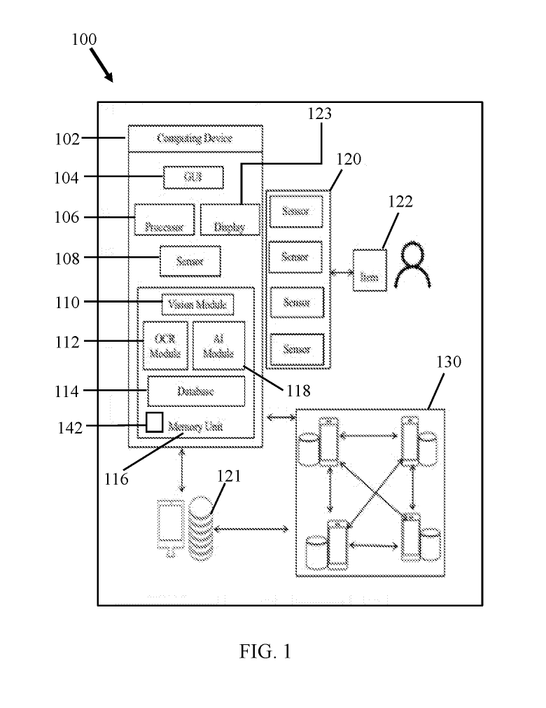

Some embodiments of this invention, illustrating all its features, will now be discussed in detail with respect to FIGS. 1 to 6.

Referring to FIG. 1 describes the block diagram representation of the system 100 including a computing device 102, a sensor unit 120, a server 121, at least one item 122, and block chain of interconnected devices. The computing device 102 further comprises a graphical user interface 104, a processor 106, a display unit 123, a sensor 108, a vision module 110, an object recognition and optical character recognition (OCR) module 112, an Artificial intelligence unit (AI) module 118, Controller module 142, a database 114, and a memory unit 116. All the components of the computing device 102 are connected to each other by means of a connecting module such as a data bus but not limited to these only.

The graphical user interface 104 is configured to receive user input via a touch input or a gesture input. The input from the user triggers the processor 106 to generate a signal to the sensor unit 120 to capture information associated with the at least one item 122.

Additionally, there is a memory unit 116 which comprises a vision module 110, OCR module 112, AI module 118, core module 142 and a database 114. The vision module 110 is configured to capture raw data related to the said item 122 via internal and the external sensor 120. The raw data relates to information about the item such as the type of item, date of manufacturing of the item, the model number of the item, the brand name of the item and the like. These should not be construed as a limitation, there could be additional information also.

In one embodiment of the present invention, the core module 142 is a software module, call it the CORE, the core module 142 hold all the other software module altogether. The core module 142 is a controller module which controls all module and their functionality. The core module 142 is adapted to trigger or activate other module based on the user input and system or device requirement. The core module 142 gives out instructions, gets back data and then gives out more instructions and also helps train and reinforce the learning of all 3 AI's i.e. vision module 110, the object recognition, optical character recognition OCR 112 and databasing and decision-making module AI. The AI's are vision module 110, the object recognition, and optical character recognition OCR 112 and databasing and decision-making module AI, the databasing AI will be checking it against various databases for various aspects that are in addition to the 2 vision AI systems. The databasing means indexing, categorizing and cataloging the primary and raw data in the database.

In one embodiment of the present invention, a Vision Module 110, OCR module 112, AI module 118, core or controller module 142 is a software agent or module which is capable of proffering all the functionality as described herein. The Vision Module 110, OCR module 112, AI module 118, core module 142 is communicably coupled to all the component of the system to perform various functionality. The Vision Module 110, OCR module 112, AI module 118, Core or controller module 142 is a software agent or module which executed or run by at least one processor. The Vision Module 110, OCR module 112, AI module 118, Core or controller module 142 are adapted to run in any computing device such mobile phone, kiosk, laptop, tablet, and processor enable computing device.

In one embodiment of the present invention, the Core module 142 is adapted to monitor the GUI 104, sensors (120,108), Vision Module 110, OCR module 112 and AI module 118 and their functionality. The Core module 142 is connected to each of the AI's (110, 112 and 118) module, processor 106, GUI 104, database 114, the sensors (120,108), block chain distributed ledger 130, server 121 and storage devices (remote database). The Core module 142 allows all the component of the system to communicate with each other and perform their functionality based on the communication.

The sensor unit 120 and the sensor 108 comprises a plurality of sensors selected from the group of a Digital Camera, Video Device, Microscopic Camera, high resolution image sensor, motion sensor, weight sensor, x-ray sensor, camera sensor, Temperature Sensor, Proximity Sensor, Accelerometer, IR Sensor (Infrared Sensor), Pressure Sensor, Light Sensor, Ultrasonic Sensor, Smoke, Gas and Alcohol Sensor, Touch Sensor, Color Sensor, Humidity sensor, Tilt Sensor, biosensor, chemical sensor, metal detector, Flow and Level Sensor, Touch Sensor, barcode reader, RFID reader. X-Ray, SEM or other sensory/validation devices and the combination thereof.

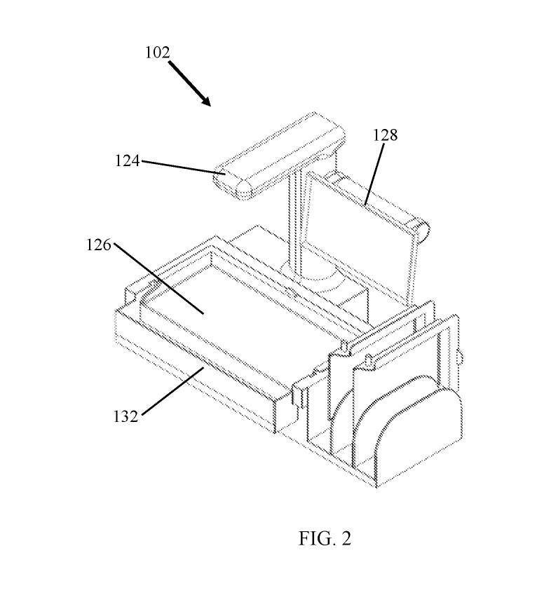

Referring to FIG. 2 shows the computing device 102 at the manufacturing unit. The computing device 102 is shown installed at the manufacturing unit for an exemplary embodiment only. The computing device 102 can be installed on each entity which is capable of taking the item under custody. The manufacturing unit is provided with a computing device 102 to scan and collect all the data related to all the items manufactured in that particular manufacturing unit. The computing device 102 comprises a processing unit 132, a camera 124, a scale 126, and a screen 128. The computing device 102 is not limited to the above-noted components only and can be designed to include a plurality of sensors to capture more sensitive data related to the product.

The vision module 110 includes a set of computer-executable instructions (i.e. a HealthSure software application) that when executed by the processor 106 cause the sensor unit 120 to capture data related associated the manufactured item 122. The piezoelectric sensor is used for weighing the items before final packaging and delivery.

In one exemplary embodiment, the size of the computing device 102 is approximately 4 inches in height, 10 inches in width, and 12 inches in length. A rear side of the computing device 102 is provided with an L bracket that allows the camera 124 and lighting to properly illuminate a base of the computing device 102. The camera 124 is configured to capture item related data and provide captured data to the vision module 110, AI module 118 of Block chain system. The item 122 related data captured by the sensor unit 120 comprises raw data.

The raw data includes data of the item captured by each sensor which includes barcode information, RFID information, images of packaging, image of the item or package, X-ray images from all side, weight of the item, items, printing pattern or colored used on the packaging, watermark information, packaging, labelling and markings, date of expiry and manufacturing of the item, ingredient information or salt used for a pharmaceutical product and the like.

In one embodiment of the present invention, the raw data relates to information about the item such as UPC code, serial number, the type of item, date of manufacturing of the item, the model number of the item, the brand name of the item and the like. These should not be construed as a limitation, there could be additional information also.

The raw data also includes information related to medical prescription prescribed by a doctor to a patient which further includes information of handwriting of the physician or doctor, a signature of the doctor and ink color etc.

The camera captures and provides the captured data to the at least one entity to identify correct medication type and quantity. The camera is also configured to confirm manufacturing quality and detect for possible counterfeiting as a standard routine. This computing device is capable of communicatively coupled to at least one electronic device present in close proximity.

The software application includes executable computer instructions that can be run from at least one electronic device selected from a group of a tablet, mobile phone, pager, laptop, computer, Kiosk or any other mobile device having processor and display.

The vision system or module 110 detects, tracks, and verifies the patient's conformity with the doctor's prescribed instructions. The status of the item can be tracked and checked by any entity based on the block chain distributed ledger 130. The supply chain information remains visible to each entity starting from the manufacturing unit, a pharmacist, a doctor, a nurse, a patient, and some law and enforcement personnel.

The item information of the scanned item 122 forms a genesis block on the block chain. The genesis block, also known as a first block, includes all the information captured by the sensor unit 120 and fingerprint data of the manufacturing unit or at first time the product is processed. The fingerprint data comprises a hash of a manufacturing unit or processing device.

In one embodiment of the invention, item 122 is a drug-related item and medical prescription. However, item 122 is not limited to the drug-related items and medical prescription only and can include any general-purpose item as well.

The information related to the item 122 is updated on the distributed ledger every time the change of custody occurs. The item 122 related information is stored in the database 114 and also in server 121.

In one embodiment of the invention, the item information can be chained together with a change in custody of the item from the manufacturer to the patient including intermediate operating units such as a pharmacist, doctor, nurse. Each time item 122 is shifted from a first holding entity to a second holding entity the change of custody occurs. At the time of change of custody, a second block is generated with the item information of the manufacturing unit and the fingerprint data of the second holding entity. The second generated block is appended to the end of the existing blockchain.

In one exemplary embodiment of the invention, each block in the blockchain includes fingerprint data of a preceding block. For example, the second block includes the fingerprint data of the first block and the third block includes fingerprint data of the second block. Accordingly, at the time the third block is added to the blockchain, it includes the fingerprint data of the second block and the hash of the genesis block. The genesis block is created by the manufacturing unit and subsequently, the first block can be generated by at least one of the pharmacist, doctor, nurse, patient or law and enforcement personnel.

The fingerprint data of each succeeding block depends on the fingerprint data of the preceding block and this arrangement makes the system more complex and minimizes the chances of counterfeit or fraudulent. The changes made in one block are identified by the other blocks and it becomes easy to track and fraud entry in the block. All the entities on the network are able to see the transaction made by all other entities and a distributed ledger associated with each device is updated after each transaction.

The OCR module 112 is configured to identify a primary data from the raw data based on at least one predefined parameter and store the primary data in the memory unit 116. The primary data is associated with the item and the AI module 118 is configured to retrieve the raw data and primary data from the memory unit 116 to generate the second fingerprint data based on the primary data and based on the at least one predefined parameter.

The Controller module 142 program is adapted to instruct the AI 118 to generate the hash or the core program can generate the hash. It will be dependent on what type of working is being recorded.

The at least one predefined parameter include item related information such as the type of item, serial number, maker information, manufacturing information, manufacturing date, serial number, expiry date, or any information related to the item.

At least one holding entity such as the manufacturer of item 122, operates the vision module 110 to execute the computer-executable instructions stored in the memory unit. The vision module 110 executes the computer-executable instructions in an event a transaction is broadcasted across the distributed computer network. At every transaction, the respective nodes in the network validate the transaction and based on the status of validity of the transaction, the transaction is provided in a first block that is further appended to the blockchain of the distributed ledger 130. Each entity in the network stores the distributed ledger that is updated at the end of every successful transaction.

In one embodiment of the invention, the patient that is a member node in the network provides the access rights to the doctor or the pharmacist by providing a public key or private key information to the respective entities. With this information, the doctor keeps track of the medication cycle and dosage of the patients efficiently.

The doctor can control the dosage by changing the prescription previously assigned to the patient. The permission granted by the patient is added to the fingerprint data for the respective block whenever a transaction is initiated by the doctor. The distributed ledger is stored at each node of the respective entities and as well as at the server 121.

In one another embodiment of the invention, the permission rights assigned to a plurality of entities are verified by the parent entity before those entities are performing any transaction. The patient grants the permission for the doctor to track, analyze or update the dosage prescribed.

The updated information includes a second block of information that further comprises the first fingerprint data and a unique fingerprint data of the handling entity.

The first fingerprint data or the unique fingerprint data is generated based on a hash of a digital representation of the at least one product, a public key of a manufacturing client who generates the record of the at least one product, and a digital signature comprising a private key of the manufacturing client.

In one embodiment of the invention the manufacturing unit having the computing device provided with the plurality of sensors to scan the product/item. the scanning includes capturing information related to barcode, manufacturing date, weight, expiry date, and unique serial number. The number of parameters is not limited to these only and can incorporate additional parameters. Once the product is out from the manufacturing unit it is provided on the supply chain. Each time the product is handled to a new charge of custody, the fingerprint information of the new user who is handling the particular products is associated with the product resulting in the updated information in the distributed ledger by using blockchain technology.

Each intervening entity can track in real time the product related information the doctor, nurse, and patient are provided with the cellular phone installed with the vision software is configured to capture a picture of the product. The processor in at least one capturing cellular phone compares the captured information with the already stored information in a storage unit in the distributed ledger.

The system generates an alert message in an event the comparison of the captured information is different from the already stored information. In this manner, the fraudulent or counterfeit drugs can be identified and also its source is identified based on the information available on distributed ledger via the blockchain technology. The system then updates the compared information on the distributed ledger.

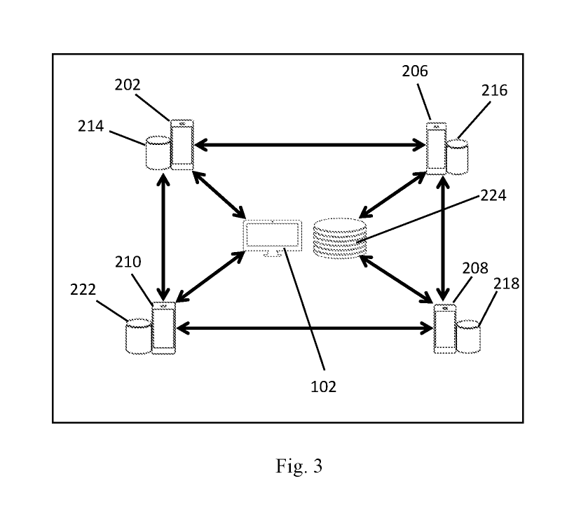

Referring to FIG. 3 is a block diagram representation of the interconnection of computing devices of a plurality of user terminals. The manufacturing unit 202, the computing device 102 associated with the manufacturing unit 202 are interconnected to the n number of devices such as the terminals associated with the patient 206, the terminal associated with the pharmacist 208, and a terminal associated with the doctor/physician 210.

Each of the plurality of terminals is associated with the computing devices to scan and analyze the items received and processed by the respective terminals. Each of the plurality of terminals having a copy of distributed ledger.

The fingerprint data is unique for each terminal and the fingerprint data 214 is associated with the manufacturing unit 202, fingerprint data 216 is associated with patient's terminal 206, the fingerprint data 218 is associated with pharmacist's terminal 208, and the fingerprint data 222 is associated with physician's terminal 210. All the communication terminals are interconnected to each other and to the centralized server 224. The ledger data updated at the time of every transaction is updated at the server 224 and a copy of the ledger is saved in the each of the plurality of the terminals.

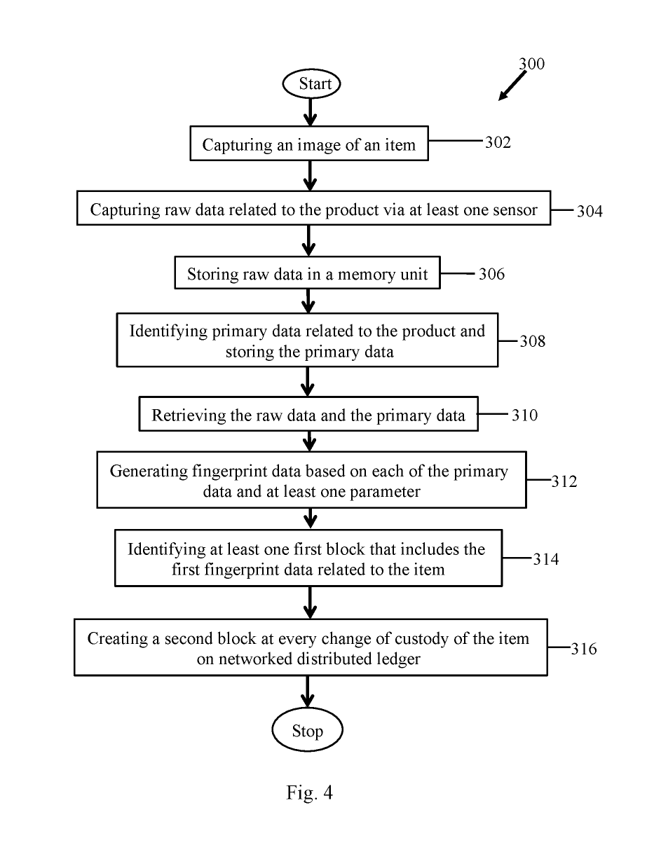

FIG. 4 is a flowchart 300 representing the steps of generation of block information on a networked distributed ledger. At step 302, upon receiving the user input, the camera module is configured to capture an image of the item. At step 304, the vision module 110 is configured to activate the sensor unit 120 to capture the raw data of the item. At step 306, the captured raw data is stored in the memory by the vision module 110.

At step 308, the system is adapted to send the raw data to OCR module 112, which is adapted to identify the item information or primary data from the raw data based on the at least one predefined parameter and store the primary data in the memory unit 116.

The identification of the primary data is achieved by identifying an object or a character marked/printed on the item. If the item or the outer packaging of the item includes characters, the OCR module 112 is configured to identify the letters or wordmarks and generates meaningful data.

Further, the OCR module 112 is connected to the database 114. The database includes pre-stored cataloged data of the image of the item and characters form a pre-stored image. The pre-stored data is collected whenever an item is processed or a transaction on the distributed ledger takes place.

The OCR module 112 is further configured to identify the region of interest based on the object and character resonation algorithm and compare with pre-stored data to identify the item and its information.

In one embodiment, whenever a new or unique image or character/object is unrecognizable, then a manual input about the image or character/object is provided by the user. That information of a new or unique image or character/object are unrecognizable is stored in the data and cataloged. So that, if in the future, the same type of character or object or image comes, the OCR module 112 will recognize it and cataloged the data. This process is also known as self-learning process, via AI module 118, which is achieved by using at least one of machine learning or deep learning software.

The item is identified based on at least one predefined parameter. The at least one predefined parameter include item related information such as the type of item, serial number, maker information, manufacturing information or any information related to the item.

At step 310, the AI module 118 is configured to retrieve the raw data and primary data from the memory unit 116. The AI module 118 is further configured to generate second fingerprint data based on the primary data and the at least one predefined parameter. The AI module is adapted to use the cataloged data stored in the memory unit 116 to create a learning database. The learning data is updated automatically whenever a new, unique data or unrecognizable data is cataloged so that the cataloged data is used in the future for identifying the information by comparing with the presorted cataloged data.

In an embodiment of the present invention, at the manufacturing unit, the AI module 118 is trained to pre-store information about every item stocked in particular manufacturing premises. It includes a huge reference library about all different shapes and sizes and other identifying characteristics that may be visible.

At step 312, the AI module 118 is further configured to generate the fingerprint data based on each of the primary data and the at least one parameter.

At step 314, the AI module 118 is further configured to identify a first block that includes the first fingerprint data related to the item in the networked distributed ledger 130.

The AI module 118 is further configured to identify at least one first block having the first fingerprint data related to the item, the first block is stored on the networked distributed ledger 130. The first fingerprint data is created at the previous change of custody point by the computing device 102 by processing the item in the same manner described above. Further, at step 316 a second block is created at every change of custody of the item on the networked distributed ledger.

The networked distributed ledger is related to a replicated, shared and synchronized digital data. The networked distributed ledger is accessible to all the points for changing the custody of the item in the supply chain management system. The AI module 118 is further configured to generate a second block at every change of custody of the item on the networked distributed ledger 130 based on the fingerprint data of the second device and the hash value of the manufacturing unit (first device). The second block including the first fingerprint data and the second fingerprint data is created at every change in custody of the item in the supply chain management.

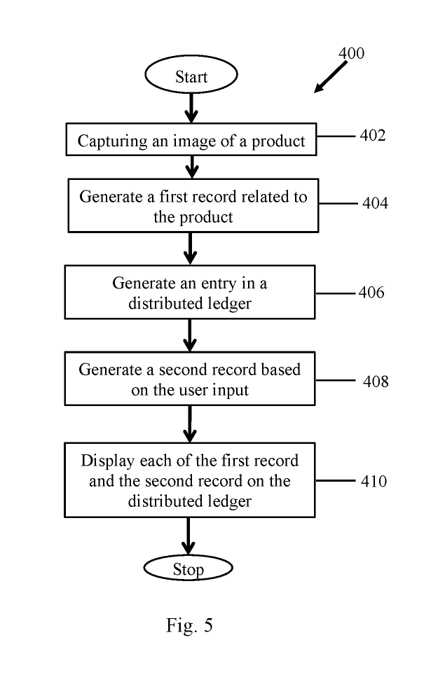

In a second embodiment of the present invention as disclosed by FIG. 5 describes a flowchart representing the steps of capturing the product related information. At step 402, the computing device 102 with the graphical user interface 104, configured to capture an image of the at least one product/item. At step 404, the computing device generates at least one first record of the at least one product based on the captured image of at least one product.

In one embodiment of the present invention, the at least one first record comprising a hash of a digital representation of the at least one product, a public key of a manufacturing client who generates the record of the at least one product, and a digital signature comprising a private key of the manufacturing client.

At step 406, a peer-to-peer network comprising one or more nodes that are in communication with the client computing device and are configured to generate an entry in a distributed ledger by performing the steps 406. Further, at step 408, at least one-second record of the at least one product based on user input. At step 410, the computing device display of each of the at least one first record and the at least one-second record on the distributed ledger.

In one exemplary embodiment of the present invention, the at least one-second record comprises a double hash of the at least one product, the public key of the manufacturing client, and a digital signature comprising a private key of the user that generates the at least one-second record. Each of the at least one first record and the at least one-second record is associated with a common blockchain.

In one exemplary embodiment of the present invention, the at least one first record is associated with a first blockchain and the at least one-second record is associated with a second blockchain.

In one exemplary embodiment of the present invention, the digital signature of the user comprising a hash of the digital signature of the manufacturing client with the double hashed at least one product and the public key of the manufacturing client.

In one exemplary embodiment of the present invention, the system generates an alarm or alert message to each of the member terminals of the network based on detection of fraud or counterfeit of the detected item. The system generates the signature information of the previous terminal under whose custody the item was held as the main reason for fraud or counterfeit of the particular item. The burden of counterfeit falls immediately to the terminal handling the product at that time of counterfeit.

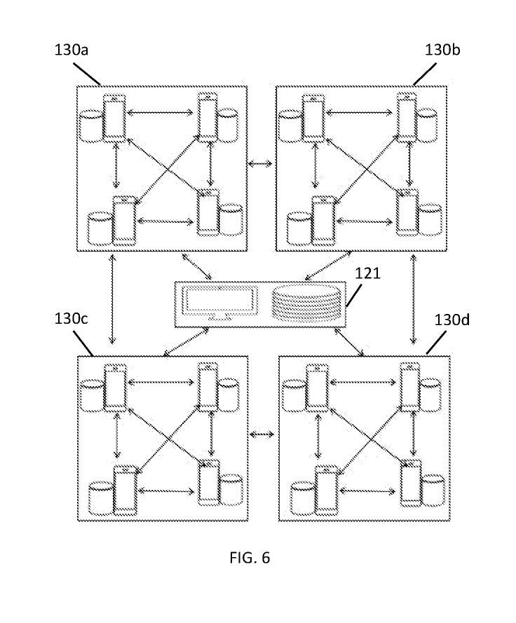

Referring to FIG. 6 discloses a system of one or more interlinked blockchain. In a third embodiment of the present invention, the system comprises a plurality of interconnected devices that are associated with different network distributed ledgers and using the blockchain technology. The plurality of devices comprises 130a to 130d that are interconnected to each other and with a common server 121. These network of devices 130a to 130d that are connected to the common server 121 and share a commonly distributed ledger. In this manner, the separate groups of devices form a cluster of one network that shares a single distributed ledger.

The system includes software to track both fixed devices as well as portable devices. The fixed devices include diagnosing hardware such as Computed tomography (CT) scan, magnetic resonance imaging (MRI) and X-ray Machines and small portable care devices like defibrillators or crutches.

The vision module includes large or small asset value recorded in various data types such as pictures, video, software, and documentation as well as equipment owner contact data. The recorded value is used to identify the counterfeit products or to find out a misplaced drug by an authority of custody who was handling it. At least one of a doctor, nurse, patient, or law enforcement personnel will be able to contact the owner in an event an item is misplaced from its supply chain.

The machine learning helps the system to create a strong database based on an intelligent script system that facilitates to read the prescription written by at least one doctor. The system is configured to read the handwritten prescription written by a doctor and compares this prescription with the other prescriptions written by the same doctor for other patients. By doing so the system builds a comprehensive data set per doctor. This comprehensive dataset helps to find out any erroneous or fraudulent prescription.

The system allows the doctors to check medications for a particular patient anytime especially for narcotics or other controlled medications that are prescribed by the doctor so that their patients not get addicted to the overdose of those prescribed drugs or the patients should not abuse those drugs.

The number of pills count is decided based on the doctor's input via a user interface of a computing device. The computing device is one of a computing device installed at each stage of the supply chain starting from the manufacturing unit to the pharmacists, nurse and at a patient terminal so that anyone can scan the item so check its authenticity. The doctors and nurses can keep track of their n number of patients simultaneously with less effort. The system monitors and tracks the medications securely and recording all the events on a distributed ledger, via a blockchain technology. The sequence of events started from the manufacturing to dispensing pharmacy to the prescribed patients. This technique provides a limited diversion of incidence and less probability of counterfeit from packaging to handling.

The system is a combination of a vision module, artificial intelligence and blockchain technology. The present system provides doctors, nurses, pharmacists, technicians, patients, and emergency law enforcement personnel to quickly and accurately identify any counterfeit drug if present in the supply chain.

The system generates an alarm to alert at least one entity associated with the supply chains to warn about the presence of fraudulent drugs. The system enhances the safety of patients located anywhere in the world.

The system aids the patients to prevent from the potentially deadly Adverse Drug Event (ADE) and Adverse Drug Reaction (ADR) through pill identification, preparation including dose detection and administrative needs by errors due to handwritten prescriptions, instructions, incorrect or fraudulent labeling.

The system allows users to track the drug dosage and checking the possible drug interactions and tracking dosage data. The system is further configured to flag possible potential dangerous or deadly drug interactions on a patient by patient basis.

The system allows the patients to share their fingerprint information with at least one doctor so that the patient's records are accessible to the doctor. The doctor can change the prescription and dosage of medicines based on the current health conditions of the patient.

The system is a combination of vision software, artificial intelligence (AI) and blockchain technology. The vision system is associated with a digital camera, an audio recording unit, and a video recording unit. The identification of the supply chain ensures that the patient should receive the right medication. Each party is able to track and see the movement of the medicines from one entity to the other. The hash value includes the detailed information of the entity handling with the product any time and it gets uploaded on the distributed ledger once the item is passed to the next entity.

In one another embodiment of the invention, this system facilitates hospitals to track medications for all patients to help ensure the utmost quality of care. This system will detect errors that can enter the manufacturing, transportation, prescribing, preparation, dosing and consumption events.

In one embodiment of the invention, the medication is produced in n number of forms including solid, semi-solid, immediate, controlled release, tablet, capsule. The produced medication is produced to its packaging that includes aspects bottles, vials, closures and blister packs along with its identifying characteristics including packaging, labeling and markings allow the pharmaceuticals to be accurately and precisely tracked through the supply chain all the way to the patient.

In one another embodiment of the invention wholesalers is able to track all medications and detect potential supply chain issues including diversion, counterfeiting, repacking, relabeling, expiration, storage, distribution and handling related defects.

In one another embodiment of the present invention the medical providers, doctors, nurses are able to easily and accurately accommodates a vast amount of patients with enhanced accuracy, transparency and accountability.

The user and or external input(s) must select which option(s) to execute in the visual component and or artificial intelligence component. This is for a newly manufactured, created, generated, produced item to be added to the blockchain it will first start by capturing input from a digital camera, video recorder, microscopic zoom, x-ray, or another input device (s) or mechanism. This action will be the start of or an addendum to a block on the blockchain component. If it is the start of a block, this record is known as the genesis event. This genesis event will capture all predefined data that has been captured by the vision system as determined by the user and or external input(s) selection. If the block already exists, which is determined by the vision components providing inputs to the artificial intelligence component that then checks the blockchain component and or relevant database, data set and or another registry; at which time an additional record referred to an addendum(s) including any or all predefined characteristics any or all analyzed, identified, discovered, generated, calculated data will then have added to the block which is part of the blockchain component.

Once the vision software has generated the information to be recorded it is then inputted in the artificial intelligence component that will then analyze it for various predetermined characteristics and or it will input additional training data to the visual component for future processing to improve or increase recognition and or throughput.

The artificial intelligence component will extract relevant data from its analysis of the input data from the visual component. After the artificial intelligence component has produced extracted data from the analysis of the vision component inputs, the artificial intelligence component will take all data and generate, calculate or compute a cryptographic hash. The cryptographic hash will store any or all relevant, identified, discovered, generated, calculated, defined and or predefined data as determined by both inputs from the visual component and inputs from the artificial intelligence component. This data can include external data from additional inputs. This data from additional inputs can include internal or external sensors external or internal data observed, analyzed, identified, discovered, generated, and calculated during the manufacture, production, testing, analysis or another process of the item and or process.

Once the cryptographic hash is generated, also known as a block; it will then be recorded and or registered on the blockchain and or relevant database, data set or another registry. The cryptographic hash is to include any and all analyzed, identified, discovered, generated, calculated data and which may or will contain any or all information as to company, operator, location, time, date and other relevant data including data related to the use of, operation of, software of, license of, security of, value of, owner of and other relevant of.

Once the cryptographic hash is recorded to the blockchain component and or relevant database, data set and or another registry the process is now complete and the record will now be available to the inquiry at any time by repeating the process as stated above.

Due to the cryptographic hash any and all analyzed, identified, discovered, generated, and or calculated data is now in a secure and protected environment.

The inventive subject matter discloses a system for detecting supply chain management, pharmaceutical counterfeit and fraudulent prescription, the system comprising: at least one computing device with a user interface, wherein the at least one computing device is configured to: capture an image of at least one product; a vision unit communicatively coupled to at least one sensor, wherein the vision unit is configured to capture raw data related to the at least one product via the at least one sensor; and store the raw data in a storage unit; an object recognition and character recognition (OCR) engine configured to: identify primary data, related to the at least one product, from the raw data based on at least one predefined parameter; and store the primary data in the storage unit; and an artificial intelligence (AI) module configured to retrieve the raw data and primary data from the storage unit; and generate a first fingerprint data based on each of the primary data and at least one predefined parameter; identify at least one first block that includes the first fingerprint data related to the at least one product, wherein the at least one product is available on a networked distributed ledger; and create a second block at every change in custody of the at least one product on networked distributed ledger, wherein the second block includes the first fingerprint data and a second fingerprint data.

In one objective of the present invention, the AI module is further configured to reject the change in custody of the at least one product in an event the first fingerprint data is not identified on the networked distributed ledger.

In one another objective of the present invention the AI module is further configured to retrieve the primary data of the at least one product from the storage unit, and compare the primary data of the at least one product with a pre-stored data related to the at least one product; and authenticate the at least one product based on the comparison of the primary data with the pre-stored data, wherein the pre-stored data comprises at least one anti-counterfeit parameter.

In one another objective of the invention, the AI module is further configured to: identify the at least one product to be a counterfeit product based on the comparison; and transmit an alert to at least one authority, wherein the alert includes information of last change of custody. The AI module, based on the detection of the counterfeit product, is further configured to generate an alarm on the computing device associated with at least one of a manufacturing unit, a doctor, a pharmacist or a patient.

In one another objective of the invention, the at least one computing device, the vision unit, the OCR engine, and the AI module are communicatively connected to each other.

In one second embodiment of the present invention, the AI module is further configured to, create a genesis block and add the first fingerprint data to the genesis block on the networked distributed ledger based on a determination that the first block is unavailable on the networked distributed ledger.

In one another embodiment of the invention, the first fingerprint data is encrypted data.

In one second embodiment of the present invention, the change in custody is shifted from at least one of a manufacturing unit to at least one of a pharmacist, a doctor, a nurse, a patient, a technician, or an emergency and law enforcement personnel. The at least one predefined parameter includes a label, a barcode, an image of the at least one product, a serial number of a manufacturing unit.

In one objective of the present invention is to provide a system and method for detecting supply chain management, pharmaceutical counterfeit and fraudulent prescription, the method comprising: capturing an image of at least one product; capturing raw data related to the at least one product via at least one sensor; storing the raw data in a storage unit; identifying primary data, related to the at least one product, from the raw data based on at least one predefined parameter; storing the primary data in the storage unit; retrieving the raw data and primary data from the storage unit; generating a first fingerprint data based on each of the primary data and at least one predefined parameter; identifying at least one first block that includes the first fingerprint data related to the at least one product, wherein the at least one product is available on a networked distributed ledger; and creating a second block at every change in custody of the at least one product on networked distributed ledger, wherein the second block includes the first fingerprint data and a second fingerprint data.

The disclosed invention is presented with reference to the accompanying drawings. The purpose served by the disclosure, however, is to provide an example of the various features and concept related to the invention and not to limit the scope of the invention.

Reasonable variations and modifications of the illustrated examples in the foregoing written specification and drawings are possible without departing from the scope of the invention. It should further be understood that to the extent the term “invention” is used in the written specification, it is not to be construed as a limited term as to number of claimed or disclosed inventions or the scope of any such invention, but as a term which has long been conveniently and widely used to describe new and useful improvements in technology.

The scope of the invention supported by the above disclosure should accordingly be construed within the scope of what it teaches and suggests to those skilled in the art.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the inventive subject matter.

What is claimed is:

1. A system for detecting pharmaceutical counterfeit and fraudulent prescription, the system comprising:
at least one computing device with a user interface, wherein the at least one computing device is configured to:
capture an image of at least one pharmaceutical product;
a vision unit communicatively coupled to at least one sensor, wherein the vision unit is configured to:
capture raw data related to the at least one pharmaceutical product via the at least one sensor, and store the raw data in a storage unit;
an object recognition and character recognition (OCR) engine configured to:
identify primary data, related to the at least one pharmaceutical product, from the raw data based on at least one predefined parameter; and
store the primary data in the storage unit; and
an artificial intelligence (AI) module configured to:
retrieve the raw data and primary data from the storage unit; and
generate a first fingerprint data based on each of the primary data and at least one predefined parameter;
identify at least one first block that includes the first fingerprint data related to the at least one pharmaceutical product, wherein the at least one pharmaceutical product is available on a networked distributed ledger; and
create a second block at every change in custody of the at least one pharmaceutical product on a networked distributed ledger, wherein the second block includes the first fingerprint data and a second fingerprint data.

2. The system as claimed in claim 1, wherein the AI module is further configured to reject the change in custody of the at least one pharmaceutical product in an event the first fingerprint data is not identified on the networked distributed ledger.

3. The system as claimed in claim 1, wherein the AI module is further configured to:
retrieve the primary data of the at least one pharmaceutical product from the storage unit; and
compare the primary data of the at least one pharmaceutical product with a pre-stored data related to the at least one pharmaceutical product; and
authenticate the at least one pharmaceutical product based on the comparison of the primary data with the pre-stored data,
wherein the pre-stored data comprises at least one anti-counterfeit parameter.

4. The system as claimed in claim 3, wherein the AI module is further configured to:
identify the at least one pharmaceutical product to be a counterfeit product based on the comparison; and
transmit an alert to at least one authority, wherein the alert includes information of last change of custody.

5. The system as claimed in claim 1, wherein the AI module is further configured to, create a genesis block and add the first fingerprint data to the genesis block on the networked distributed ledger based on a determination that the first block is unavailable on the networked distributed ledger.

6. The system as claimed in claim 1, wherein the first fingerprint data is encrypted data.

7. The system as claimed in claim 1, wherein the change in custody is shifted from at least one of a manufacturing unit to at least one of a pharmacist, a doctor, a nurse, a patient, a technician, or an emergency and law enforcement personnel.

8. The system as claimed in claim 1, wherein the at least one predefined parameter includes a label, a barcode, an image of the at least one pharmaceutical product, a serial number of a manufacturing unit, ingredient information, expiry date and manufacturing date of the at least one pharmaceutical product.

9. The system as claimed in claim 4, wherein, based on the identification of the counterfeit pharmaceutical product, the AI module is further configured to generate an alarm on a computing device associated with at least one of a manufacturing unit, a doctor, a pharmacist or a patient.

10. The system as claimed in claim 1, wherein the at least one computing device, the vision unit, the OCR engine, and the AI module are communicatively connected to each other.

11. A system for detecting pharmaceutical counterfeit and fraudulent prescription, the system comprising:
at least one computing device with a user interface, wherein the at least one computing device is configured to:
capture an image of at least one medical prescription;
a vision unit communicatively coupled to at least one sensor, wherein the vision unit is configured to:
capture raw data related to the at least one medical prescription via at the least one sensor, and store the raw data in a storage unit;
an object recognition and character recognition (OCR) engine configured to:
identify primary data, related to the at least one medical prescription, from the raw data based on at least one predefined parameter; and
store the primary data in the storage unit; and
an artificial intelligence (AI) module configured to:
retrieve the raw data and primary data from the storage unit; and
generate a first fingerprint data based on each of the primary data and at least one predefined parameter;
identify at least one first block that includes the first fingerprint data related to the at least one medical prescription, wherein the at least one medical prescription is available on a networked distributed ledger; and
create a second block at every change in custody of the at least one medical prescription on a networked distributed ledger, wherein the second block includes the first fingerprint data and a second fingerprint data.

12. The system as claimed in claim 11, wherein the AI module is further configured to reject the change in custody of the at least one medical prescription in an event the first fingerprint data is not identified on the networked distributed ledger.

13. The system as claimed in claim 11, wherein the AI module is further configured to:
retrieve the primary data of the at least one medical prescription from the storage unit; and
compare the primary data of the at least one medical prescription with a pre-stored data related to the at least one medical prescription; and
authenticate the at least one medical prescription based on the comparison of the primary data with the pre-stored data,
wherein the pre-stored data comprises at least one anti-counterfeit parameter.

14. The system as claimed in claim 13, wherein the AI module is further configured to:
identify the at least one medical prescription to be a counterfeit medical prescription based on the comparison; and
transmit an alert to at least one authority, wherein the alert includes information of last change of custody.

15. The system as claimed in claim 11, wherein the at least one predefined parameter includes a label, a barcode attached to the at least one medical prescription, a doctor's handwriting, ink color, doctors signature on the at least one medical prescription.

16. A method for identifying a pharmaceutical counterfeit and fraudulent prescription, the method comprising:
capturing an image of at least one product;
capturing raw data related to the at least one product via at least one sensor;
storing the raw data in a storage unit;
identifying primary data, related to the at least one product, from the raw data based on at least one predefined parameter;
storing the primary data in the storage unit;
retrieving the raw data and primary data from the storage unit;
generating a first fingerprint data based on each of the primary data and at least one predefined parameter;
identifying at least one first block that includes the first fingerprint data related to the at least one product, wherein the at least one product is available on a networked distributed ledger; and
creating a second block at every change in custody of the at least one product on the networked distributed ledger, wherein the second block includes the first fingerprint data and a second fingerprint data.

17. The method as claimed in claim 16, further comprising rejecting the change in custody of the at least one product in an event the first fingerprint data is not identified on the networked distributed ledger.

18. The method as claimed in claim 16, further comprising:
retrieving the primary data of the at least one product from the storage unit; and
comparing the primary data of the at least one product with a pre-stored data related to the at least one product; and
authenticating the at least one product based on the comparison of the primary data with the pre-stored data,
wherein the pre-stored data comprises at least one anti-counterfeit parameter.

19. The method as claimed in claim 18, further comprising:
identifying the at least one product to be a counterfeit product based on the comparison; and
transmitting an alert to at least one authority, wherein the alert includes information of the last change of custody.

20. A non-transitory computer-readable medium to store computer-executable instructions, executed by a processor, cause a computer to perform detection of supply chain management, the method comprising:
capturing an image of at least one product;
capturing raw data related to the at least one product via at least one sensor;
storing the raw data in a storage unit;
identifying primary data, related to the at least one product, from the raw data based on at least one predefined parameter;
storing the primary data in the storage unit;
retrieving the raw data and primary data from the storage unit;
generating a first fingerprint data based on each of the primary data and at least one predefined parameter;
identifying at least one first block that includes the first fingerprint data related to the at least one product, wherein the at least one product is available on a networked distributed ledger; and
creating a second block at every change in custody of the at least one product on a networked distributed ledger, wherein the second block includes the first fingerprint data and a second fingerprint data.
